(19) United States
(12) Patent Application Publication
(10) Pub. No.: US 2014/0323943 A1
(43) Pub. Date: Oct. 30, 2014

(54) Dialysis device and method for the operation thereof

(71) Applicant: Werner Kleophas, Kaarst (DE)

(72) Inventor: Werner Kleophas, Kaarst (DE)

(21) Appl. No.: 14/166,384

(22) Filed: Jan. 28, 2014

Related U.S. Application Data
(63) Continuation of application No. PCT/EP2012/064868, filed on Jul. 30, 2012.

(30) Foreign Application Priority Data
Jul. 28, 2011 (DE) 10 2011 052 264.6

(51) Int. Cl. A61M 1/36

(52) U.S. Cl. CPC A61M 1/3639
USPC 604/6.09; 604/257; 604/246; 604/151

(57) ABSTRACT

A dialysis device according to the invention includes a hemodialyzer which is connectable to the blood circulation of a patient, a plasma storage device for temporary storage of blood plasma of the patient and a supply device for supplying blood plasma stored in the plasma storage device into the blood circulation of the patient

The invention relates to a dialysis device and a method for operating a dialysis device.

Problems encountered during operation of known dialysis devices include uncontrolled blood pressure drops in the treated patients. Such blood pressure drops result in particular from the fact that the volume changes associated with the withdrawal of liquid during the dialysis are poorly tolerated by the treated patients especially in the case of advanced blood vessel paralysis (vasoparalysis).

In order to overcome these problems, diverse therapy approaches were developed. From WO 2005/1 18024 A1 a hemodialysis device is known in which various counter measures are automatically taken such as switching off the ultrafiltration or administering a circulation-stabilizing agent, when a drop of blood pressure is recognized.

From WO 2010/004400 A2 the monitoring of volume balances during dialysis is known, wherein the dialysis operation is automatically controlled on the basis of the detected volume changes.

From JP 2004358269 A a dialysis device is known in which the blood pressure is to be controlled by adjusting the sodium dosage.

In praxis known therapy approaches, in particular the treatment involving certain volume or sodium profiles, have proven to be often insufficiently effective, wherein in some cases the time point of a critical blood pressure drop is merely delayed or advanced.

In view of the above it is an object of the present invention to provide a dialysis device and a method for operating the same, which enable effectively preventing or ameliorating a problematic blood pressure drop during the dialysis treatment.

This object is solved by the device according to the features of the independent patent claim 1 or the method according to the features of the alternative independent patent claim 6.

A dialysis device according to the invention includes:
a hemodialyzer which can be connected to the blood circulation of a patient;
a plasma storing device for temporary storage of blood plasma of the patient; and
a supply device for supplying plasma which is stored in the plasma storage device into the blood circulation of the patient.

According to an embodiment, the dialysis device also has a plasma separator for separating blood plasma from the blood of the patient, wherein the plasma separator is connected to the plasma storage device.

According to an embodiment, the supply device has a valve device via which the plasma separator can be selectively connected to the blood circulation of the patient.

According to an embodiment, the supply device has a plasma pump for supplying blood plasma, which is stored in the plasma storage device, into the blood circulation of the patient.

The invention also relates to a method for operating a dialysis device, wherein the dialysis device includes:
a hemodialyzer which can be connected to the blood circulation of a patient;
a plasma storage device for temporary storage of blood plasma of the patient; and
a supply device for supplying blood plasma stored in the plasma storage device into the blood circulation of the patient;
wherein the supply device is controlled based on the actual value of the intradialytic liquid volume.

According to an embodiment, the supply device is controlled so that th the blood plasma stored in the plasma storage device is supplied into the blood circulation of the patient at a time point at which the intradialytic liquid volume during the hemodialysis reaches a volume value V2 which corresponds to a physiological hydration state.

Further embodiments of the invention are set forth in the description and the dependent claims.

Generally the hypervolaemia (=increase of the volume of the circulating blood, i.e., the blood present in the circulation) of the dialysis patient is determined by the increase of liquid in the interdialytic interval.

For bridging the dialysis-free interval of typically 1 to 2 days between the dialysis treatments, the so-called dry weight is adjusted at the end of the dialysis treatment, in which the liquid volume has to be unphysiologically low in order to provide sufficient leeway for the expected liquid increase. From this volume value (in the following referred to as V1) the volume increases to a value V2 up to the day following the dialysis treatment, which value V2 corresponds to a physiological hydration state. On the day prior to the next scheduled dialysis treatment an unphysiologically high volume, in the following referred to as V3, is reached which corresponds to a hyper-hydration. This volume V3 which corresponds to the hyper-hydration in turn results in a strongly increased hydrostatic pressure difference because due to the given neuropathy practically no vascular constriction or dilation occurs.

During the hemodialysis, ultrafiltration leads to a decrease of the volume below this volume value V3—which corresponds to the hyper-hydration. It should be noted in this context, however, that this volume V3 includes an intravascular as well as an intracellular component. In the ultrafiltration, initially only the intravascular component is accessed, whereas the intracellular liquid components in the volume V3 first have to flow back in. This process of flowing back in is generally referred to as “refilling”.

Beside the hydrostatic pressure difference, the course of this refilling process depends to a great degree on the colloid osmotic pressure difference (also referred to as oncotic pressure difference), which in turn is significantly influenced by the protein components of the plasma. On the other hand the osmotic pressure difference due to sodium ions or the like only plays a minor role for the refilling.

A circulatory collapse typically occurs when the volume drops from the value V3 via the value V2 to the value V1, which corresponds to hypovolaemia.

The invention is based on the concept to perform a substitution using the own plasma of the patient, which was separated beforehand, namely preferably at the time point at which during the hemodialysis or the ultrafiltration the volume value V2 is reached. This enables stabilizing circulation and preventing a circulatory collapse. The approach according to the invention is based on the recognition that it can be assumed that a decrease of the colloid osmotic pressure due to a separated protein amount VPROT is tolerable at the beginning of the dialysis treatment or has no circulation-destabilizing effect, which in particular is due to the fact that the decrease of the volume 5 at the beginning of the dialysis treatment from the value V3 to the value V2 promotes refilling and with this stabilizes circulation.

The refilling process is in particular promoted by increasing the colloid osmotic pressure difference, which results from the reinfusion of the own plasma. This measure has a more direct effect on the refilling compared to influencing the osmotic pressure differences by adjusting the sodium dosage, as attempted in the state of the art without any remarkable success.

In the following, the invention is explained in more detail with reference to the included drawing.

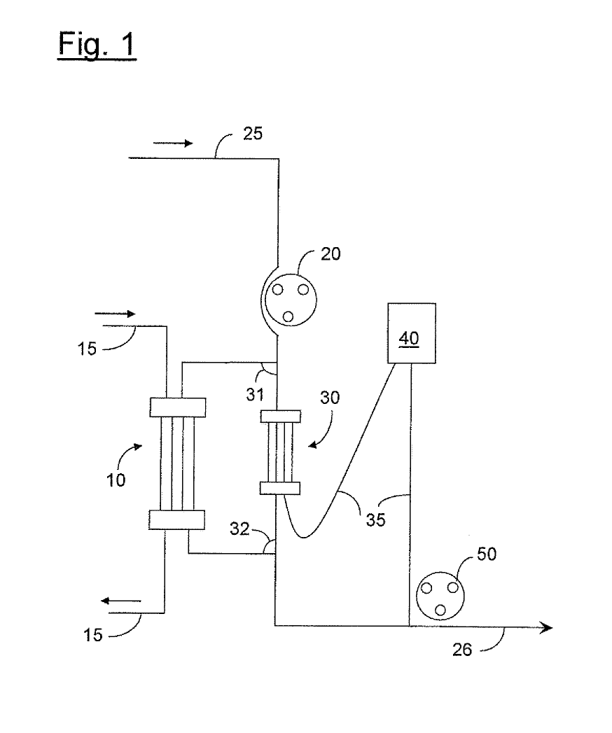

The sole FIG. 1 shows a schematic representation for explaining the construction and the functioning of the dialysis device according to an embodiment of the invention.

As schematically shown in FIG. 1, a dialysis device according to the invention has as is known a hemodialyzer 10, which is arranged in a dialysis line 15, and in which the blood of the patient undergoing the dialysis is purified via a rinsing fluid, which flows over the opposite side of a membrane present in the hemodialyzer and which is supplied and discharged in the dialysis line 15, based on the concentration difference of the substances contained in the blood. For maintaining the blood flow, a blood pump 20 is connected, also in a known manner, during operation between an arterial blood line 25 and a venous blood line 26.

In the dialysis device according to the invention, a plasma separator 30 is connected in the region of the venous blood line 26. From this plasma separator 30 a plasma line 35 leads via a plasma storage device 40 and a plasma pump 50 up to the venous blood line 26. Upstream and downstream of the plasma separator 30 a respective (two-way) valve 31 or 32 is provided, via which the blood flow, which is supplied by the blood pump 20, is selectively conducted via the plasma separator 30 and the plasma storage device 40. Thus the valve device formed by the valves 31, 32, the plasma pump 50 and the plasma line 35 together form a supply device for supplying blood plasma stored in the plasma storage device into the blood circulation of the patient.

As an alternative, the connection of the plasma separator 30 can also occur in the region of the arterial blood line 26, i.e., upstream of the hemodialyzer 10.

By means of the arrangement according to the invention, the blood plasma which is separated at the beginning of the dialysis treatment, can be temporarily stored, wherein this blood plasma is then supplied during the dialysis treatment preferably at a time point at which during the hemodialysis or the ultrafiltration the volume value V2 is reached which corresponds to the physiological hydration state. The supply device is thus controlled based on the actual value of the intradialytic liquid volume.

For this, the blood flow is guided at the beginning of the dialysis treatment via the valve 31 over the plasma separator 30, whereby the blood which is separated there is stored in the plasma storage device 40 and the plasma-depleted blood remaining after the separation is supplied via the valve 32 to the venous blood line 26. In a further (not shown) embodiment the plasma-depleted blood, which remains after the separation, can also be supplied to the hemodialyzer 10.

The separation process is preferably started at the beginning of the dialysis process or shortly thereafter, i.e., at a time point at which a hyper-hydration is present with a corresponding volume V3. Further the separation process is preferably performed within a relatively short time period—compared to the overall duration of the dialysis process—for example within the first half hour of an overall four-hour dialysis process, i.e., preferably within about the first eighth of the available dialysis time. The refilling process, which is supported by the own plasma reinfusion, is to be able to occur over an as long as possible time period, if possible before the unphysiological hydration state V1 is reached.

However, it is also within the scope of the present invention to perform the separation continuously up to or until shortly after reaching the state V2. The amount of own plasma to be separated is limited among other things by the fact that the physiological hydration state V2 should not already be fallen below by the initial separation process.

It is further within the scope of the present invention to be able to perform the reinfusion of the previously separated own plasma at any time point of the ongoing dialysis treatment in order to account for the individual circulatory situation of the patient. This means the reinfusion can be performed at any time for stabilizing the circulation and for promoting the refilling process, for prophylaxis of a circulatory instability, preferably when reaching a physiological hydration state V2 and for therapy preferably when reaching a rather unphysiologically low hydration state V1.

For example the reinfusion can preferably be started at a time point within the last third of the dialysis process.

When the reinfusion is performed for acute therapeutic reasons (for example due to circulatory instability) the reinfusion rate can be selected relatively high to achieve a fast therapeutic effect. However, when the reinfusion occurs without concrete therapeutic reason within the scope of the concept according to the invention, the reinfusion rate can be selected relatively low and can for example be adjusted so that the reinfusion is completed together with the dialysis treatment.

At the end of the separation process the plasma separator 30 is blocked via the valve 31 so that the blood flow only takes place via the hemodialyzer 10, whereas no pump pressure applies on the plasma line 35. For example at the time point at which during the hemodialysis or the ultrafiltration approximately a volume value V2 corresponding to the physiological hydration state is reached, the blood plasma stored in the plasma storage device 40 is supplied to the venous blood line 26 by reopening the valve 31 to supply the initially separated blood plasma to the blood circulation of the patient and to compensate the further decrease of the liquid volume. The supply of the colloid osmotically effective own plasma promotes the refilling, i.e., the flowing back of extravascular liquid into the intravascular space, thereby achieving a circulation-stabilizing effect.

The appropriate time point for starting the supply of the stored blood plasma (for example approximately at the volume value V2) is preferably determined based on the liquid homeostasis of the patient by taking among other factors the actual ultrafiltration amount and the actual circulatory condition into account.

The process control of the device according to the invention, including the control of the valves 31, 32 and the plasma pump 50, occurs preferably by means of an automated, microcomputer-supported process control, which can also initiate an early reinfusion of the stored blood plasma if needed by monitoring the monitor values of the patient (for example blood pressure).

As a result this achieves stabilizing the circulation and enables preventing a circulatory collapse of the patient due to drop in blood pressure during the dialysis treatment. In addition other liquid withdrawal symptoms such as nausea, vomiting and muscle cramps can be avoided or ameliorated.

1. Dialysis device, comprising a hemodialyzer (10) connectable to the blood circulation of a patient;
a plasma storage device (40) for temporary storing blood plasma of the patient; and
a supply device (31, 32, 35, 50) for supplying blood plasma stored in the plasma storage device (40) into the blood circulation of the patient.

2. Dialysis device according to claim 1,
characterized in that
the latter further has a plasma separator (30) for separating blood plasma form the blood of the patient, wherein the plasma separator (30) is connected to the plasma storage device (40).

3. Dialysis device according to claim 2,
characterized in that
the supply device has a valve device (31, 32) via which the plasma separator (30) can be selectively connected to the blood circulation of the patient.

4. Dialysis device according to one of the claims 1 to 3,
characterized in that
the supply device has a plasma pump (50) for supplying blood plasma stored in the plasma storage device (40) into the blood circulation of the patient.

5. Dialysis device according to one of the claims 1 to 4,
characterized in that
the supply device has a trigger device which triggers a supply of the blood stored in the plasma storage device (40) into the blood circulation of the patient in correlation to the intradialytic liquid volume during the hemodialysis, namely preferably approximately when the intradialytic liquid volume reaches a volume value (V2) which corresponds to a physiological hydration state.

6. Method for operating a dialysis device, wherein the dialysis device has:
a hemodialyzer (10) which is connectable to the blood circulation of a patient;
a plasma storage device (40) for temporary storage of blood plasma of the patient; and
a supply device (31, 32, 35, 50) for supplying blood plasma stored in the plasma storage device (40) into the blood circulation of the patient,
wherein the supply device (31, 32, 35, 50) is controlled based on the actual value of the intradialytic liquid volume and/or depending on the actual circulation condition of the patient.

7. Method according to claim 6,
characterized in that
the supply device (31, 32, 35, 50) is controlled so that blood stored in the plasma storage device (4) is supplied into the circulation of the patient either
at a time point at which during the hemodialysis the intradialytic liquid volume approximately reaches a volume value (V2) which corresponds to a physiological hydration state or
when reaching the volume value (V1) at the end of the treatment at which a rather unphysiologically low hydration state is reached or at any other time point of the ongoing dialysis therapy in response to the situation of the patient.
